(19) United States
(12) Patent Application Publication
(10) Pub. No.: US 2015/0132800 A1
(43) Pub. Date: May 14, 2015

(54) Trichoderma Hydrophobin Production

(71) Applicant: Danisco US Inc., Palo Alto, CA (US)

(72) Inventor: Michael Ward, San Francisco, CA (US)

(73) Assignee: DANISCO US INC., Palo Alto, CA (US)

(21) Appl. No.: 14/402,270

(22) PCT Filed: May 21, 2013
(86) PCT No.: PCT/US2013/042007

§ 371 Date: Nov. 19, 2014

Related U.S. Application Data
(60) Provisional application No. 61/649,654, filed on May 21, 2012.

(51) Int. Cl. C07K 14/37

(52) U.S. Cl. CPC C07K 14/37
USPC 435/69.1

(57) ABSTRACT

The invention relates to a method for maximizing expression of a biosurfactant, such as hydrophobin, in a microorganism, in particular a Trichoderma production host.

### RELATED APPLICATIONS AND INCORPORATION BY REFERENCE

This application claims benefit of priority from U.S. Provisional Application Ser. No. 61/649,654, filed 21 May 2012 and is incorporated by reference herein in its entirety.

Reference is made to international patent application Serial No. PCT/US2009/046783 filed 9 Jun. 2009, which published as PCT Publication No. WO 2009/152176 on 17 Dec. 2009, Serial No. PCT/US2010/044964 filed 10 Aug. 2010, which published as PCT Publication No. WO 2011/019686 on 17 Feb. 2011, Serial No. PCT/US2010/044964 filed 10 Aug. 2010, Serial No. PCT/US12/31104 filed 29 Mar. 2012 and Serial No. PCT/US12/33728 filed 16 Apr. 2012 and U.S. patent application Ser. No. 13/433,036 filed 28 Mar. 2012.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates to methods for producing hydrophobin in a Trichoderma production host.

### BACKGROUND OF THE INVENTION

Hydrophobins (HBFs) are small, secreted proteins of about 70 to 150 amino acids which occur in filamentous fungi, for example Schizophyllum commune. They usually have eight cysteine residues. Hydrophobins can be isolated from natural sources, but can also be obtained by means of genetic engineering methods (see, e.g., WO 2006/082251 and WO 2006/131564).

Hydrophobins may be organized in a water-insoluble form on the surface of various fungal structures, such as e.g. aerial hyphae, spores, fruiting bodies. The genes for hydrophobins could be isolated from ascomycetes, deuteromycetes and basidiomycetes. Some fungi have more than one hydrophobin gene, e.g. Schizophyllum commune, Coprinus cinereus, Aspergillus nidulans. Different hydrophobins are evidently involved in different stages of fungal development. The hydrophobins here are presumably responsible for different functions (van Wetter et al., 2000, Mol. Microbiol., 36, 201-210; Kershaw et al. 1998, Fungal Genet. Biol, 1998, 23, 18-33).

Hydrophobins identified to date are generally classed as either class I or class II. Both types have been identified in fungi as secreted proteins that self-assemble at hydrophobilic interfaces into amphipathic films. Assemblages of class I hydrophobins are generally relatively insoluble whereas those of class II hydrophobins (HBF II) readily dissolve in a variety of solvents.

As biological function for hydrophobins, besides the reduction in the surface tension of water for the generation of aerial hyphae, imparting hydrophobicity to spores is also described (Wosten et al. 1999, Curr. Biol., 19, 1985-88; Bell et al. 1992, Genes Dev., 6, 2382-2394). Furthermore, hydrophobins serve to line gas channels in fruiting bodies of lichen and as components in the recognition system of plant surfaces by fungal pathogens (Lugones et al. 1999, Mycol. Res., 103, 635-640; Hamer & Talbot 1998, Curr. Opinion Microbiol., volume 1, 693-697).

Previously, hydrophobins were prepared only with moderate yield and purity using customary time-consuming protein-chemical purification (such as size exclusion or ion-exchange column purification or HPLC) and isolation methods (such as salt precipitation and crystallization). Attempts at providing larger amounts of hydrophobins with the aid of genetic methods have not been always been successful. Production yield of HFB II in Trichoderma is low, for example, about 0.24 g/l or even lower. Therefore, there is a need in the art for a more effective method of producing hydrophobins, in particular HFB II.

Citation or identification of any document in this application is not an admission that such document is available as prior art to the present invention.

### SUMMARY OF THE INVENTION

This invention stems, in part, from Applicants' surprising and unexpected finding that hydrophobin expression in a Trichoderma production host, under the control of the cbh1 promoter and in a medium supplemented with glucose and sophorose resulted in a higher yield of hydrophobin than previously observed. Because glucose was known to downregulate cbh1 expression and lactose was known to induce cbh1 expression, lactose was previously used for inducing hfb2 production (see, e.g., Bailey et al., Appl Microbiol Biotechnol. 2002 May;58(6):721-7).

The present invention relates to a method of producing hydrophobin II in Trichoderma reesei. The method may comprise cloning a hydrophobin II (hfb2) coding sequence into an expression plasmid and transforming a Trichoderma reesei with the expression plasmid. Advantageously, the cbh1 coding sequence in the Trichoderma reesei may be deleted or disrupted.

The method may further comprise selecting a stable transformant expressing hydrophobin II. The stable transformant may be fermented in a broth containing glucose/sophorose. Optionally, an antifoaming agent may be added.

The hydrophobin II may be isolated by removing it from the cells, such as by lysis and/or filtration.

Advantageously, hydrophobin II may be produced at a concentration of above 2 g/L or above 5 g/L of the broth.

Mention is especially made of the use of “consisting essentially of” and “consists essentially of” to distinguish over, to any extent available as art, U.S. Pat. No. 7,713,725 and any document equivalent thereto, e.g., by way of subject matter and/or patent law (e.g., by being or claiming priority from or being in the same family as WO 2004/035070) and U.S. Pat. No. 7,883,872. Mention is especially made of the use of “consisting essentially of” and “consists essentially of” to distinguish over, to any extent available as art, in particular, publications by VTT Biotechnology such as, but not limited to, Nakari-Setälä et al., Eur J Biochem. 1997 Sep. 1;248(2):415-23; Ilmén et al., Appl Environ Microbiol. 1997 Apr;63(4):1298-306 and Bailey et al., Appl Microbiol Biotechnol. 2002 May;58(6):721-7.

In one embodiment, a method for producing a hydrophobin encoded by a gene under the control of an inducible promoter comprising the steps of: (a) generating a first mixture comprising between about 5% to about 75% glucose and a cellulase preparation; (b) incubating the first mixture at a temperature and for a sufficient time to produce an inducing feed composition comprising sophorose in a concentration ranging from 2 g/L to 25 g/L, gentiobiose in a concentration ranging from 35 g/L to 60 g/L, and glucose; and (c) culturing a host cell comprising a nucleotide sequence encoding a hydrophobin under the control of a sophorose-inducible promoter or a gentiobiose-inducible promoter with said inducing feed composition, in an amount effective to induce the production of hydrophobin. In another embodiment, hydrophobin is heterologous hydrophobin. In another embodiment, hydrophobin is hydrophobin I or hydrophobin II. In another embodiment, a cell is genetically engineered to express a hydrophobin gene under the control of a sophorose-inducible promoter or a gentiobiose-inducible promoter. In one embodiment, the cell is a filamentous fungal cell, preferably selected from the group consisting of Trichoderma, Humicola, Fusarium, Aspergillus, Neurospora, Penicillium, Cephalosporium, Myceliophthora, Thermomyces, Chrysosporium, more preferably Trichoderma spp., and preferably Trichoderma reesei. In one embodiment, the first mixture is incubated at about 50° C. to about 70° C., for a period of about eight hours to about seven days. In some embodiments, the hydrophobin gene is operably linked to the cbh1, cbh2, egl1 or egl2 promoter whereby the expression of hydrophobin is under the control of the cbh1, cbh2 or egl1, egl2 promoter. In another embodiment, the endogenous cellulase genes are deleted or disrupted. In another embodiment, endogenous eg15 gene is deleted or disrupted. In some embodiments a method for producing a hydrophobin encoded by a gene under the control of a glucose- and/or sophorose-inducible promoter is provided, wherein the glucose is present in the inducing feed composition in an amount comprising about 60% wt/wt, the sophorose is present in the inducing feed composition in an amount comprising about 12 g/L, the nucleic acid molecule encoding the hydrophobin is a hfb2 coding sequence, the hfb2 coding sequence is operably linked to the cbh1 promoter whereby hydrophobin expression is under the control of the cbh1 promoter, and the Trichoderma comprises a Trichoderma reesei having one or more of Trichoderma cbh1, cbh2, egl1, eg15 and egl2 coding sequences deleted or disrupted

These and other embodiments are disclosed or are obvious from and encompassed by, the following Detailed Description.

### DETAILED DESCRIPTION OF THE INVENTION

As used herein, a “biosurfactant” or a “biologically produced surfactant” pertains to a substance that decreases surface tension, such as the interfacial tension between water and a hydrophobic liquid, or between water and air, and that may be produced or obtained from a biological system. A biosurfactant or biologically produced surfactant may be a protein, advantageously a hydrophobin. The biosurfactant may occur naturally or it may be a mutagenized or genetically engineered variant not found in nature.

As used herein, a “biological system” comprises or is derived from a living organism such as a microbe, a plant, a fungus, an insect, a vertebrate or a life form created by synthetic biology. The living organism can be a variant not found in nature that is obtained by classical breeding, clone selection, mutagenesis and similar methods to create genetic diversity, or it can be a genetically engineered organism obtained by recombinant DNA technology. The living organism can be used in its entirety or it can be the source of components such as organ culture, plant cultivars, suspension cell cultures, adhering cell cultures or cell free preparations.

The biological system may or may not contain living cells when it sequesters the biosurfactant. The biological system may be found and collected from natural sources, it may be farmed, cultivated or it may be grown under industrial conditions. The biological system may synthesize the biosurfactant from precursors or nutrients supplied or it may enrich the biosurfactant from its environment.

As used herein, “production” relates to manufacturing methods for the production of chemicals and biological products, which includes, but is not limited to, harvest, collection, compaction, exsanguination, maceration, homogenization, mashing, brewing, fermentation, recovery, solid liquid separation, cell separation, centrifugation, filtration (such as vacuum filtration), formulation, storage or transportation.

As used herein, a “fermentation broth composition” refers to cell growth medium that contains a protein of interest, such as hydrophobin. The cell growth medium may include cells and/or cell debris, and may be concentrated. An exemplary fermentation broth composition is hydrophobin-containing, ultrafiltration-concentrated fermentation broth. Microfiltration is conventionally used to retain cell debris and pass proteins, e.g., for cell separation, while ultrafiltration is conventionally used to retain proteins and pass solutes, e.g., for concentration.

As used herein, the terms “polypeptide” and “protein” are used interchangeably to refer to polymers of any length comprising amino acid residues linked by peptide bonds. The conventional one-letter or three-letter code for amino acid residues is used herein. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

As used herein, a “culture solution” is a liquid comprising a biosurfactant and other soluble or insoluble components from which the biosurfactant of interest is intended to be recovered. Such components include other proteins, non-proteinaceous impurities such as cells or cell debris, nucleic acids, polysaccharides, lipids, chemicals such as antifoam, flocculants, salts, sugars, vitamins, growth factors, precipitants, and the like. A “culture solution” may also be referred to as “protein solution,” “liquid media,” “diafiltered broth,” “clarified broth,” “concentrate,” “conditioned medium,” “fermentation broth,” “lysed broth,” “lysate,” “cell broth,” or simply “broth.” The cells, if present, may be bacterial, fungal, plant, animal, human, insect, synthetic, etc.

As used herein, the term “recovery” refers to a process in which a liquid culture comprising a biosurfactant and one or more undesirable components is subjected to processes to separate the biosurfactant from at least some of the undesirable components, such as cells and cell debris, other proteins, amino acids, polysaccharides, sugars, polyols, inorganic or organic salts, acids and bases, and particulate materials.

As used herein, a “biosurfactant product” refers to a biosurfactant preparation suitable for providing to an end user, such as a customer. Biosurfactant products may include formulation excipients such as buffers, salts, preservative, reducing agents, sugars, polyols, surfactants, and the like, that are added or retained in order to prolong the functional shelf-life or facilitate the end use application of the biosurfactant.

As used herein, functionally and/or structurally similar biosurfactants are considered to be “related biosurfactants.” Such biosurfactants may be derived from organisms of different genera and/or species, or even different classes of organisms (e.g., bacteria and fungus). Related biosurfactants also encompass homologs determined by primary sequence analysis, determined by tertiary structure analysis, or determined by immunological cross-reactivity.

As used herein, the term “derivative biosurfactant” refers to a protein-based biosurfactant which is derived from a biosurfactant by addition of one or more amino acids to either or both the N- and C-terminal end(s), substitution of one or more amino acids at one or a number of different sites in the amino acid sequence, and/or deletion of one or more amino acids at either or both ends of the protein or at one or more sites in the amino acid sequence, and/or insertion of one or more amino acids at one or more sites in the amino acid sequence. The preparation of a biosurfactant derivative may be achieved by modifying a DNA sequence which encodes for the native protein, transformation of that DNA sequence into a suitable host, and expression of the modified DNA sequence to form the derivative protein.

Related (and derivative) biosurfactants include “variant biosurfactant.” Variant protein-based biosurfactants differ from a reference/parent biosurfactant, e.g., a wild-type biosurfactant, by substitutions, deletions, and/or insertions at small number of amino acid residues. The number of differing amino acid residues may be one or more, for example, 1, 2, 3, 4, 5, 6, 7, 8, 9, 10, 15, 20, 30, 40, 50, or more amino acid residues. Variant biosurfactants share at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or even at least about 99%, or more, amino acid sequence identity with a wildtype biosurfactant. A variant biosurfactant may also differ from a reference biosurfactant in selected motifs, domains, epitopes, conserved regions, and the like.

As used herein, “chimera” or “chimeric” refers to a single composition, advantageously a polypeptide, possessing multiple components, which may be from different organisms. As used herein, “chimeric” is used to refer to tandemly arranged moieties, including a biosurfactant or a variant biosurfactant thereof, which is engineered to result in a fusion protein possessing regions corresponding to the functions or activities of the individual protein moieties.

As used herein, the term “analogous sequence” refers to a sequence within a protein-based biosurfactant that provides similar function, tertiary structure, and/or conserved residues as the biosurfactant. For example, in epitope regions that contain an alpha-helix or a beta-sheet structure, the replacement amino acids in the analogous sequence preferably maintain the same specific structure. The term also refers to nucleotide sequences, as well as amino acid sequences. In some embodiments, analogous sequences are developed such that the replacement amino acids result in a variant enzyme showing a similar or improved function. In some embodiments, the tertiary structure and/or conserved residues of the amino acids in the biosurfactant are located at or near the segment or fragment of interest. Thus, where the segment or fragment of interest contains, for example, an alpha-helix or a beta-sheet structure, the replacement amino acids preferably maintain that specific structure.

As used herein, the term “homologous biosurfactant” refers to a biosurfactant that has similar activity and/or structure to a reference biosurfactant. It is not intended that homologs necessarily be evolutionarily related. Thus, it is intended that the term encompass the same, similar, or corresponding biosurfactant(s) (i.e., in terms of structure and function) obtained from different organisms. In some embodiments, it is desirable to identify a homolog that has a quaternary, tertiary and/or primary structure similar to the reference biosurfactant.

The degree of homology between sequences may be determined using any suitable method known in the art (see, e.g., Smith and Waterman (1981) Adv. Appl. Math. 2:482; Needleman and Wunsch (1970) J. Mol. Biol., 48:443; Pearson and Lipman (1988) Proc. Natl. Acad. Sci. USA 85:2444; programs such as GAP, BESTFIT, FASTA, and TFASTA in the Wisconsin Genetics Software Package (Genetics Computer Group, Madison, WI); and Devereux et al. (1984) Nucleic Acids Res. 12:387-395).

For example, PILEUP is a useful program to determine sequence homology levels. PILEUP creates a multiple sequence alignment from a group of related sequences using progressive, pair-wise alignments. It can also plot a tree showing the clustering relationships used to create the alignment. PILEUP uses a simplification of the progressive alignment method of Feng and Doolittle, (Feng and Doolittle (1987) J. Mol. Evol. 35:351-360). The method is similar to that described by Higgins and Sharp (Higgins and Sharp (1989) CABIOS 5:151-153). Useful PILEUP parameters including a default gap weight of 3.00, a default gap length weight of 0.10, and weighted end gaps. Another example of a useful algorithm is the BLAST algorithm, described by Altschul et al. (Altschul et al. (1990) J. Mol. Biol. 215:403-410; and Karlin et al. (1993) Proc. Natl. Acad. Sci. USA 90:5873-5787). One particularly useful BLAST program is the WU-BLAST-2 program (See, Altschul et al. (1996) Meth. Enzymol. 266:460-480). Parameters “W,” “T,” and “X” determine the sensitivity and speed of the alignment. The BLAST program uses as defaults a word-length (W) of 11, the BLOSUM62 scoring matrix (See, Henikoff and Henikoff (1989) Proc. Natl. Acad. Sci. USA 89:10915) alignments (B) of 50, expectation (E) of 10, M′S, N′-4, and a comparison of both strands.

As used herein, the phrases “substantially similar” and “substantially identical,” in the context of at least two nucleic acids or polypeptides, typically means that a polynucleotide or polypeptide comprises a sequence that has at least about 70% identity, at least about 75% identity, at least about 80% identity, at least about 85% identity, at least about 90% identity, at least about 91% identity, at least about 92% identity, at least about 93% identity, at least about 94% identity, at least about 95% identity, at least about 96% identity, at least about 97% identity, at least about 98% identity, or even at least about 99% identity, or more, compared to the reference (i.e., wild-type) sequence. Sequence identity may be determined using known programs such as BLAST, ALIGN, and CLUSTAL using standard parameters. (See e.g., Altschul, et al. (1990) J. Mol. Biol. 215:403-410; Henikoff et al. (1989) Proc. Natl. Acad. Sci. USA 89:10915; Karin et al. (1993) Proc. Natl. Acad. Sci USA 90:5873; and Higgins et al. (1988) Gene 73:237-244). Software for performing BLAST analyses is publicly available through the National Center for Biotechnology Information. Also, databases may be searched using FASTA (Pearson et al. (1988) Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution. Another indication that two nucleic acid sequences are substantially identical is that the two molecules hybridize to each other under stringent conditions (e.g., within a range of medium to high stringency).

As used herein, “wild-type” and “native” biosurfactants are those found in nature. The terms “wild-type sequence,” and “wild-type gene” are used interchangeably herein, to refer to a sequence that is native or naturally occurring in a host cell. In some embodiments, the wild-type sequence refers to a sequence of interest that is the starting point of a protein engineering project. The genes encoding the naturally-occurring protein may be obtained in accord with the general methods known to those skilled in the art. The methods generally comprise synthesizing labeled probes having putative sequences encoding regions of the biosurfactant, preparing genomic libraries from organisms expressing the protein, and screening the libraries for the gene of interest by hybridization to the probes. Positively hybridizing clones are then mapped and sequenced.

The methods of the present invention can be applied to the isolation of a biosurfactant from a culture solution. Advantageously, the biosurfactant is a soluble extracellular biosurfactant that is secreted by microorganisms.

A group of exemplary biosurfactants are the hydrophobins, a class of cysteine-rich polypeptides expressed by filamentous fungi. Hydrophobins are small (˜100 amino acids) polypeptides known for their ability to form a hydrophobic coating on the surface of objects, including cells and man-made materials. First discovered in Schizophyllum commune in 1991, hydrophobins have now been recognized in a number of filamentous fungi. Based on differences in hydropathy and other biophysical properties, hydrophobins are categorized as being class I or class II.

The expression of hydrophobin conventionally requires the addition of one or more antifoaming agents (i.e., antifoam) during fermentation. Otherwise, the foam produced by hydrophobin polypeptides may saturate breather filters, contaminate vents, cause pressure build-up, and may reduce protein yield. As a result, crude concentrates of hydrophobin conventionally contain residual amounts of antifoam, as well as host cell contaminants, which are undesirable in a hydrophobin preparation, particularly when the hydrophobin is intended as a food additive. Advantageously, the antifoam may be silicone-based polymers and/or non-silicone organics.

Hydrophobin can reversibly exist in forms having an apparent molecular weight that is greater than its actual molecular weight, which make hydrophobin well suited for recovery using the present methods. Liquid or foam containing hydrophobin can be continuously or periodically harvested from a fermentor for protein recovery as described, or harvested in batch at the end of a fermentation operation.

The hydrophobin can be any class I or class II hydrophobin known in the art, for example, hydrophobin from an Agaricus spp. (e.g., Agaricus bisporus), an Agrocybe spp. (e.g., Agrocybe aegerita), an Ajellomyces spp., (e.g., Ajellomyces capsulatus, Ajellomyces dermatitidis), an Aspergillus spp. (e.g., Aspergillus arvii, Aspergillus brevipes, Aspergillus clavatus, Aspergillus duricaulis, Aspergillus ellipticus, Aspergillus flavus, Aspergillus fumigatus, Aspergillus fumisynnematus, Aspergillus lentulus, Aspergillus niger, Aspergillus unilateralis, Aspergillus viridinutans), a Beauveria spp. (e.g., Beauveria bassiana), a Claviceps spp. (e.g., Claviceps fusiformis), a Coccidioides spp., (e.g., Coccidioides posadasii), a Cochliobolus spp. (e.g., Cochliobolus heterostrophus), a Crinipellis spp. (e.g., Crinipellis perniciosa), a Cryphonectria spp. (e.g., Cryphonectria parasitica), a Davidiella spp. (e.g., Davidiella tassiana), a Dictyonema spp. (e.g., Dictyonema glabratum), an Emericella spp. (e.g., Emericella nidulans), a Flammulina spp. (e.g., Flammulina velutipes), a Fusarium spp. (e.g., Fusarium culmorum), a Gibberella spp. (e.g., Gibberella moniliformis), a Glomerella spp. (e.g., Glomerella graminicola), a Grifola spp. (e.g., Grifola frondosa), a Heterobasidion spp. (e.g., Heterobasidion annosum), a Hypocrea spp. (e.g., Hypocrea jecorina, Hypocrea lixii, Hypocrea virens), a Laccaria spp. (e.g., Laccaria bicolor), a Lentinula spp. (e.g., Lentinula edodes), a Magnaporthe spp. (e.g., Magnaporthe oryzae), a Marasmius spp. (e.g., Marasmius cladophyllus), a Moniliophthora spp. (e.g., Moniliophthora perniciosa), a Neosartorya spp. (e.g., Neosartorya aureola, Neosartorya fennelliae, Neosartorya fischeri, Neosartorya glabra, Neosartorya hiratsukae, Neosartorya nishimurae, Neosartorya otanii, Neosartorya pseudofischeri, Neosartorya quadricincta, Neosartorya spathulata, Neosartorya spinosa, Neosartorya stramenia, Neosartorya udagawae), a Neurospora spp. (e.g., Neurospora crassa, Neurospora discreta, Neurospora intermedia, Neurospora sitophila, Neurospora tetrasperma), a a Ophiostoma spp. (e.g., Ophiostoma novo-ulmi, Ophiostoma quercus), a Paracoccidioides spp. (e.g., Paracoccidioides brasiliensis), a Passalora spp. (e.g., Passalora fulva), Paxillus filamentosus Paxillus involutus), a Penicillium spp. (e.g., Penicillium camemberti, Penicillium chrysogenum, Penicillium marneffei), a Phlebiopsis spp. (e.g., Phlebiopsis gigantea), a Pisolithus (e.g., Pisolithus tinctorius), a Pleurotus spp., (e.g., Pleurotus ostreatus), a Podospora spp. (e.g., Podospora anserina), a Postia spp. (e.g., Postia placenta), a Pyrenophora spp. (e.g., Pyrenophora tritici-repentis), a Schizophyllum spp. (e.g., Schizophyllum commune), a Talaromyces spp. (e.g., Talaromyces stipitatus), a Trichoderma spp. (e.g., Trichoderma asperellum, Trichoderma atroviride, Trichoderma viride, Trichoderma reesei [Hypocrea jecorina]), a Tricholoma spp. (e.g., Tricholoma terreum), a Uncinocarpus spp. (e.g., Uncinocarpus reesii), a Verticillium spp. (e.g., Verticillium dahliae), a Xanthodactylon spp. (e.g., Xanthodactylon flammeum), a Xanthoria spp. (e.g., Xanthoria calcicola, Xanthoria capensis, Xanthoria ectaneoides, Xanthoria flammea, Xanthoria karrooensis, Xanthoria ligulata, Xanthoria parietina, Xanthoria turbinata), and the like. Hydrophobins are reviewed in, e.g., Sunde, M et al. (2008) Micron 39:773-84; Linder, M. et al. (2005) FEMS Microbiol Rev. 29:877-96; and Wosten, H. et al. (2001) Ann. Rev. Microbiol. 55:625-46.

In a particularly advantageous embodiment, the hydrophobin is from a Trichoderma spp. (e.g., Trichoderma asperellum, Trichoderma atroviride, Trichoderma viride, Trichoderma reesei (Hypocrea jecorina)), advantageously Trichoderma reesei.

Hydrophobin-like proteins (e.g. “chaplins”) have also been identified in filamentous bacteria, such as Actinomyces and Streptomyces sp. (WO01/74864; Talbot, 2003, Curr. Biol, 13: R696-R698). These bacterial proteins by contrast to fungal hydrophobins, may form only up to one disulphide bridge since they may have only two cysteine residues. Such proteins are an example of functional equivalents to hydrophobins, and another type of molecule within the ambit of biosurfactants of methods herein.

Fermentation to produce the biosurfactant is carried out by culturing the host cell or microorganism in a liquid fermentation medium within a bioreactor or fermenter. The composition of the medium (e.g. nutrients, carbon source etc.), temperature and pH are chosen to provide appropriate conditions for growth of the culture and/or production of the biosurfactant. Air or oxygen-enriched air is normally sparged into the medium to provide air/oxygen to the culture.

As used herein, a “fermentation broth composition” refers to cell growth medium that contains a protein of interest, such as hydrophobin. The cell growth medium may include cells and/or cell debris, and may be concentrated. An exemplary fermentation broth composition is hydrophobin-containing, ultrafiltration-concentrated fermentation broth. Microfiltration is conventionally used to retain cell debris and pass proteins, e.g., for cell separation, while ultrafiltration is conventionally used to retain proteins and pass solutes, e.g., for concentration.

Advantageously, a cross-flow membrane filtration recovery method may allow for a preparation of a hydrophobin concentration as described in PCT Patent Publication WO 2011/019686 which is incorporated by reference. In other embodiments, size exclusion filtration and crystallization may also allow for a preparation of a hydrophobin concentrate.

The present invention relates particularly to the expression of a surfactant, advantageously hydrophobin, more advantageously hydrophobin 2 in a production system, advantageously Trichoderma, more advantageously Trichoderma reesei. The hydrophobin II gene (hfb2) encodes a secreted protein of 86 amino acids. After cleavage of the signal sequence (15 amino acids), the mature protein (HFBII) contains 71 amino acids with 4 disulfide bonds. Its molecule weight is about 7 kd in size.

A sequence of the hfb2 gene may be obtained from the EMBL Data Bank under the accession number Y11894. In particular, the sequence of the hfb2 gene may be CACATTCACTCAACTCCTCTTTCTCAACTCTCCAAACACAAACATTCTTTGTTGAATA CCAACCATCACCACCTTTCAAGATGCAGTTCTTCGCCGTCGCCCTCTTCGCCACCAG CGCCCTGGCTGCTGTCTGCCCTACCGGCCTCTTCTCCAACCCTCTGTGCTGTGCCACC AACGTCCTCGACCTCATTGGCGTTGACTGCAAGACCCGTATGTTGAATTCCAATCTC TGGGCATCCTGACATTGGACGATACAGTTGACTTACACGATGCTTTACAGCTACCAT CGCCGTCGACACTGGCGCCATCTTCCAGGCTCACTGTGCCAGCAAGGGCTCCAAGC CTCTTTGCTGCGTTGCTCCCGTGGTAAGTAGTGCTCGCAATGGCAAAGAAGTAAAA AGACATTTGGGCCTGGGATCGCTAACTCTTGATATCAAGGCCGACCAGGCTCTCCTG TGCCAGAAGGCCATCGGCACCTTCTAAAGCAATGGCTTGCTTTACTGCCGGCAGTCT TTGAGAACTCTGGGCTCACAAAAGACGACTTGCATGTATCATGGGGGCTCGCAAAT GGGAGGATTTGGAGGGGATTGAGGCTGGGTTTGGCCTATTAGAGGATTGCATAATG GAAGATTTGCGAGCAGGACATAGACGTATCTAGAGTTCTAGT (SEQ ID NO: 1) wherein the exons are nucleotides 81-210, 281-366 and 439-483 and the introns are nucleotides 211-280 and 367-438. The translated hydrophobin may have the sequence MQFFAVALFATSALAAVCPTGLFSNPLCCATNVLDLIGVDCKTPTIAVDTGAIFQAHCA SKGSKPLCCVAPVADQALLCQKAIGTF (SEQ ID NO: 2). The hfb2 coding sequence may be ascertained by one of skill in the art when presented with an hfb2 nucleotide sequence. The hfb2 coding sequence may be expressed in Trichoderma, advantageously Trichoderma reesei.

Advantageously, the expression of the surfactant may be under the control of a promoter, advantageously a cellulase promoter, in particular, an exo-cellobiohydrolase promoter, an endoglucanase promoter, or a beta-glucosidase promoter. In a particularly advantageous embodiment, the promoter is a cbh1 promoter. In a particularly advantageous embodiment, the expression of hfb2 is under the control of a cbh1 promoter and terminator. In other embodiments, the promoter may be and/or include a cbh2, egl1, or egl2, promoter (see, e.g., US Patent Publication 20100323426).

The expression vector containing and expressing hfb2 may contain selectable markers, such as, but not limited to, an amdS marker for the selection of fungal transformants and the ColE1 ori and AmpR gene for E. coli manipulations. The hfb2 coding sequence may also then be transferred to an expression plasmid for suitable expression in Trichoderma, advantageously Trichoderma reesei. For example, the expression plasmid may be pTrex3gM.

The vector pTrex3gM has been previously described, see for example, U.S. Patent Application Publication Nos. 20110136197, 2010216682 or 20100041104. Briefly, the vector is based on an E coli vector including an origin of replication and a gene conferring ampicillin resistance. It was engineered to become a Gateway destination vector (Hartley, J. L. et al., (2000) Genome Research 10: 1788-1795) to allow insertion using Gateway technology (Invitrogen) of any desired open reading frame between the promoter and terminator regions of the T. reesei cbh1 gene. The Aspergillus nidulans amdS gene was inserted for use as a selectable marker in transformation.

The hfb2 open reading frame is inserted into the expression plasmid, using the Gateway system, between the cbh1 promoter and terminator regions. The expression plasmid may then be transformed to a production host, advantageously a Trichoderma production host, more advantageously a Trichoderma reesei production host.

For example, a biolistic transformation of T. reesei with a construct of the present invention may be performed using the following protocol. A suspension of spores (approximately 3.5×10⁸ spores/ml from a P-37 derived strain of T. reesei is prepared. Between 100 μl -200 μl of this spore suspension is spread onto the center of plates of MM acetamide medium. MM acetamide medium had the following composition: 0.6 g/L acetamide; 1.68 g/L CsCl; 20 g/L glucose; 20 g/L KH₂PO₄; 0.6 g/L CaCl₂.2H₂O; 1 ml/L 1000× trace elements solution; 20 g/L Noble agar; pH 5.5. 1000× trace elements solution contained 5.0 g/l FeSO₄.7H₂O, 1.6 g/l MnSO₄.H₂O, 1.4 g/l ZnSO₄.7H₂O and 1.0 g/l CoCl₂.6H₂O. The spore suspension was allowed to dry on the surface of the MM acetamide medium in a sterile hood.

Transformation of T. reesei may be performed using a Biolistic® PDS-1000/He Particle Delivery System from Bio-Rad (Hercules, Calif.) following the manufacturer's instructions (Lorito, M. et al., 1993, Curr Genet 24:349-56). 60 mg of M10 tungsten particles were placed in a microcentrifuge tube. 1 mL of ethanol is added, the mixture is briefly vortexed and allowed to stand for 15 minutes. The particles are centrifuged at 15,000 rpm for 15 mins. The ethanol is removed and the particles are washed three times with sterile dH₂O before 1 mL of 50% (v/v) sterile glycerol was added. After ten seconds of vortexing to suspend the tungsten, 25 μl of tungsten/glycerol particle suspension was removed and placed into a microcentrifuge tube.

While continuously vortexing the 25 μl tungsten/glycerol particle suspension, the following may be added in order, allowing 5′ incubations between additions; 2 μl (100-300 ng/μl) expression vector (a cut fragment), 25 μl of 2.5M CaCl₂ and 10 μl of 0.1 M spermidine. After a 5′ incubation post spermidine addition, the particles are centrifuged for 3 seconds. The supernatant is removed; the particles are washed with 200 μl of 70% (v/v) ethanol and then centrifuged for 3 seconds. The supernatant is removed; the particles are washed with 200 μl of 100% ethanol and centrifuged for 3 seconds. The supernatant is removed and 24 μl 100% ethanol is added and mixed by pipetting. The tube is placed in an ultrasonic cleaning bath for approximately 15 seconds to further resuspend the particles in the ethanol. While the tube is in the ultrasonic bath, 8 μl aliquots of suspended particles are removed and placed onto the center of macrocarrier disks that were placed into a desiccator.

Once the tungsten/DNA solution is dried onto the macrocarrier (approximately 15′), it is placed into the bombardment chamber. Next a plate containing MM acetamide with spores and the bombardment process is performed using 1100 psi rupture discs according to the manufacturers' instructions. After the bombardment of the plated spores with the tungsten/DNA particles, the plates are incubated at 28° C. Large transformed colonies are picked to fresh secondary plates of MM acetamide after 5 days (Penttila et al., (1987) Gene 61:155-164) and incubated another 3 days at 28° C. Colonies which show dense, opaque growth on secondary plates are transferred to individual MM acetamide plates. These are grown another three days and transferred to potato dextrose agar plates (PDA) and allowed to incubate another 7-10 days at 28° C. to allow sporulation.

In a particularly advantageous embodiment, the production host may comprise one or more deletions and/or mutations of cellulase genes, advantageously the cbh1 gene. In one embodiment, the production host may be a cbh1 deleted production strain. In another embodiment, the production host may be a cbh1 , cbh2, egl1, and egl2 deleted production strain. In yet another embodiment, the production host cells may be the host cells described in U.S. patent application Ser. No. 13/276,467. The transformants are fermented in a shake flask in media containing glucose/sophorose.

In an advantageous embodiment, the media containing glucose/sophorose may be made from purified glucose and/or purified sophorose. Advantageously, about 1%, about 2%, or about 5% (wt/wt) glucose is present in the final solution. Advantageously, sophorose is present about 1 g/L, about 2 g/L, about 5 g/L, about 10 g/L, about 15 g/L, about 20 g/L, about 25 g/L, about 30 g/L, about 35 g/L, about 40 g/L, about 45 g/L or about 50 g/L.

In an alternative embodiment, it was discovered (see, e.g., U.S. Pat. No. 7,713,725) that when a whole cellulase preparation is added to a concentrated glucose solution, and the composition is incubated for at least two days at about 50° C. to about 75° C., preferably about 50° C. to 65° C., a sugar mixture containing appreciable quantities of an inducer of cellulase gene expression is made, i.e., the inducing feed composition. The inducing feed composition has between about 2 and 25 g/L sophorose. In addition, the inducing feed composition may have between about 35 and 60 g/L gentiobiose. Surprisingly, the resulting mixture does not need any further purification. It is competent to induce cellulase production as is. This discovery provides the inexpensive alternative to lactose or purified sophorose that is needed, as well as a less cumbersome alternative to solid cellulose for the production of proteins regulated by inducible promoters in a filamentous fungus. It is specifically contemplated that the inventive composition is useful for production of cellulase or inducing genes under the control of cellulase promoter in Trichoderma.

In an alternative method of producing the inducing feed composition, the end fermentation broth (whole cellulase plus cells) may be added to a glucose solution (e.g., 20% v/v). The presence of the cells does not affect sophorose formation. Thus, there is no need to use a recovered cellulase (i.e., a cellulase preparation isolated from the cells). The enzyme mixture present at the end of fermentation may be used although the cells are still present.

In one embodiment, the invention provides a composition comprising a concentrated glucose solution and whole cellulase preparation that may be used as an inducing feed for the production of a protein of interest (such as a surfactant, advantageously hydrophobin, more advantageously hydrophobin II) by a filamentous fungus.

In one embodiment an inducing feed may be made by preparing a sterile solution of 5%-75% (wt/wt) glucose. A whole cellulase preparation from Trichoderma reesei is added to a sterile glucose solution to a final concentration of between 2 g and 20 g total protein/L. The final protein range may be as low as 0.5 g/L and as high as 50 g/L. The solution is incubated at 50° C.-75° C., preferably between 50° C. and 65° C. The solution is incubated for between 8 hours and 7 days with mixing. In one embodiment the incubation period is greater than two days. In second embodiment the incubation period is two days. In third embodiment the incubation period is three days. The final sterile solution is harvested and used for fermentation feeding. In one embodiment the inducing feed is prepared with a 60% (wt/wt) glucose solution. In another embodiment the inducing feed is prepared by adding whole cellulase preparation to the glucose solution to a final concentration of about 2 to about 10 g total protein/L.

A glucose/sophorose feed may be produced as described in U.S. Pat. No. 7,713,725. For example, 60% (wt/wt) glucose solution may be dissolved and sterilized for 30 minutes at 121° C. The temperature was decreased to 65° C. and 10 g of total protein (whole cellulase previously produced by T. reesei)/L is added. The mixture is agitated slowly and held at 65° C. for 3 days. The sophorose content was measured at 12 g/L in this 60% glucose solution.

The fermentations are advantageously performed with growth about 25° C. to about 37° C., advantageously at about 34° C. and about pH 3 to about pH 5.5, advantageously about pH 3.5, and production at about 25° C. to 30° C., advantageously at about 28° C. and about pH 3 to about pH 5.5, advantageously at about pH 4.5. Antifoam may be added. In another embodiment, it may be advantageous to insolubilize the hydrophobin to avoid foaming (see, e.g., U.S. provisional patent application Ser. No. 61/469,067.

In an advantageous embodiment, a stock solution of about 60% glucose (wt/wt) may react with a stock solution of about 12 g/L sophorose, wherein the mixture may be slowly fed into a fermenter. The mixture may be continuously fed into a fermentor tank. Each of the streams introduced into the reactor preferably is controlled at a predetermined rate, or in response to a need determinable by monitoring such as concentration of the carbon and energy substrate, pH, dissolved oxygen, oxygen or carbon dioxide in the off-gases from the fermentor, cell density measurable by light transmittancy, or the like. The feed rates of the various materials can be varied so as to obtain as rapid a cell growth rate as possible, consistent with efficient utilization of the carbon and energy source, to obtain as high a yield of microorganism cells relative to substrate charge as possible. See, e.g., U.S. Pat. No. 7,713,725. In an advantageous embodiment, the feed rate may be about 5 to about 20 grams of dry solids per liter per hour, advantageously about 10 grams of dry solids per liter per hour.

Because harvest of the fermentor may produce a broth that consisted predominantly of foam, the fermentors are advantageously cooled and depressurized very slowly. The broth may also need to be diluted with about 50%, about 100%, about 150%, about 200%, about 250%, about 300%, about 350% or about 400% warm process water to help disperse the foam. Cells may be removed by filtration, such as a 40×40 cm filter press, using diatomaceous earth Celite FW-12 as admix. The filtrate may be displaced with warm process water. This method gives full recovery of hydrophobin in a homogeneous, clear filtrate with insignificant amounts of foam. Since the temperature is above the cloudpoint of the antifoaming agent, this helped reduce the amount of antifoam in the filtrate.

Ultrafiltration (UF) concentration of the filtrate may be performed below the cloudpoint of the MAZU® antifoam, again to aid in further removal of the antifoam. The UF element may be a Millipore PES 10 kD 0.6 m2 spiral wound cartridge connected to a rotary vane recirculation pump and a cooled reservoir.

Two Liters of concentrate is prepared for sterile filtration by adding 20 g of diatomaceous earth filter aid (Filtercel-70). Sterile filtration may be performed through two 1-Liter Millipore 0.2 μm PES bottle top membrane filters used in parallel with a partial vacuum to avoid foam-out from dissolved air in the filtrate.

In a particularly advantageous embodiment, the resultant concentration of hydrophobin may be above 1 g/L, above 2 g/L, above 3 g/L, above 4 g/L, or above 5 g/L.

Although the present invention and its advantages have been described in detail, it should be understood that various changes, substitutions and alterations can be made herein without departing from the spirit and scope of the invention as defined in the appended claims.

The present invention will be further illustrated in the following Example which is given for illustration purposes only and are not intended to limit the invention in any way.

### EXAMPLES

Hydrophobin II.

This Example reports production of the Trichoderma native hydrophobin II in the production host T. reesei. The hfb2 gene was amplified from genomic DNA of Trichoderma reesei by PCR as a 451 by DNA fragment containing three exons and two introns and with the addition of CACC at the 5′ end to facilitate directional cloning into pENTR/D-TOPO according to the directions of the vendor (Invitrogen, Carlsbad, Calif.). The hfb2 gene expression in Trichoderma was under the control of the cbh1 promoter and terminator in plasmid pTrex3gM. A quad deleted strain of T. reesei (Δcbh1, Δcbh2, Δegl1, Δeg12) as described in international patent publication WO 05/001036 may be utilized for the present invention.

Expression Plasmid.

The hfb2 expression plasmid contains an amdS marker for the selection of fungal transformants and the ColE1 ori and AmpR gene for E. coli manipulations. The PCR fragment of the hfb2 gene was first cloned into a pENTR/D-TOPO vector. The fidelity of the hfb2 gene was verified by DNA sequencing. The hfb2 gene was then transferred to pTrex3gM by Gateway cloning method to create the expression plasmid: pTrex3gM-HFBII.

Strains.

The expression plasmid was transformed to a Trichoderma production host (Morph 1.1 pyr+strain) using a biolistic transformation method using whole plasmid. The entire plasmid was inserted into genome of the Morph1.1 strain without removing the bacterial DNA sequence. Eleven stable transformants were obtained and all produced some level of HFBII. Three transformants produced significant amounts of HFBII in shake flasks. The best transformant was selected for further fermentation study.

HFBII Production in 14 L Fermentor.

0.8 L of media was inoculated with 1.5 mL Trichoderma reesei frozen spore suspension as a seed flask. This flask was split into two 0.4 L portions and transferred to 2×7 L of fermentation media in two different 15 L Biolafitte fermentors after 48 hours. The fermentor was run initially at 34° C., pH 3.5 followed by 28° C., pH 4.5. Agitation was held at 500 RPM with 10 standard liters per minute airflow. Antifoam and glucose/sophorose were fed separately. Protein production was measured by gel electrophoresis.

The fermentors were harvested by first slowly cooling and depressurizing. Harvest broth was diluted with 200% warm process water to disperse foam. Cells were removed by filtration on a 40×40 cm filter press, using diatomaceous earth Celite FW-12. The filtrate was displaced with warm process water. This method gave full recovery of HFBII in a homogeneous, clear filtrate with insignificant amounts of foam. Since the temperature was above the cloudpoint of the antifoaming agent, this helped reduce the amount of antifoam in the filtrate.

Ultrafiltration (UF) concentration of the filtrate was performed below the cloudpoint of the antifoam, to aid in further removal of the antifoam. The UF element was a Millipore PES 10 kD 0.6 m2 spiral wound cartridge connected to a rotary vane recirculation pump with a cooled reservoir. Two liters of concentrate were prepared for sterile filtration by adding 20 g of diatomaceous earth (Filtercel-70). Sterile filtration was performed through two 1-liter Millipore 0.2 μm PES bottle top membrane filters.

The final filtrate contains 95-115 g HFBII per kg of filtrate, as determined by SDS-PAGE densitometry, using lysozyme as a standard. The sterile filtrate contains 19% dry solids, which makes the enzyme overall 50-60% pure. The non-HFBII bands on the SDS-gel amount to 5% of the overall density in the lane.

Matrix-assisted laser desorption/ionization time-of-flight mass spectrometry (MALDI-TOF/MS) analyses of the hydrophobin are carried out.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. A method for producing a hydrophobin encoded by a gene under the control of an inducible promoter comprising the steps of:
(a) generating a first mixture comprising between about 5% to about 75% glucose and a cellulase preparation;
(b) incubating the first mixture at a temperature and for a sufficient time to produce an inducing feed composition comprising sophorose in a concentration ranging from 2 g/L to 25 g/L, gentiobiose in a concentration ranging from 35 g/L to 60 g/L, and glucose; and
(c) culturing a host cell comprising a nucleotide sequence encoding a hydrophobin under the control of a sophorose-inducible promoter or a gentiobiose-inducible promoter with said inducing feed composition, in an amount effective to induce the production of hydrophobin.

2. (canceled)

3. The method of claim 1, wherein the hydrophobin is hydrophobin I or hydrophobin II.

4-6. (canceled)

7. The method of claim 1, wherein the promoter is cbh1 promoter from Trichoderma reesei.

8. The method of claim 1, wherein the hydrophobin gene is under the control of a sophorose-inducible promoter.

9. The method of claim 1, wherein the protein of interest is under the control of a gentiobiose-inducible promoter.

10. The method of claim 1, wherein the cell is a filamentous fungal cell.

11. The method of claim 10, wherein the filamentous fungus is selected from the group consisting of Trichoderma, Humicola, Fusarium, Aspergillus, Neurospora, Penicillium, Cephalosporium, Myceliophthora, Thermomyces, Chrysosporium.

12. (canceled)

13. The method of claim 12, wherein the filamentous fungus is Trichoderma reesei.

14. The method of claim 1, wherein the cellulase preparation in said first mixture comprises from about 0.5 g/L to about 50 g/L total protein.

15. The method of claim 14, wherein the total protein concentration in said first mixture ranges from about 2 g/L to about 10 g/L.

16. The method of claim 1, wherein the first mixture is incubated at about 50° C. to about 70° C.

17. The method of claim 16, wherein the first mixture is incubated for between 8 hours and 7 days.

18. The method of claim 1, wherein the cellulase preparation is a Trichoderma reesei cellulase preparation.

19. (canceled)

20. The method of claim 1, wherein the first mixture is incubated at a temperature of about 65° C. for a period of two to three days.

21. The method of claim 1, in which said cellulase preparation is the product of Trichoderma reesei that has been engineered to overexpress beta-glucosidase.

22. The method of claim 1, wherein the cellulase preparation is a whole cellulase composition or a beta-glucosidase enriched cellulase composition.

23. (canceled)

24. The method of claim 21, wherein the Trichoderma cell is further modified to disrupt or delete eg15 gene.

25. The method of claim 1, wherein the hydrophobin gene is operably linked to the cbh1, cbh2, egl1 or egl2 promoter whereby the expression of hydrophobin is under the control of the cbh1, cbh2 or egl1, egl2 promoter.

26. The method of claim 1 wherein the nucleic acid molecule encoding the hydrophobin comprises an hfb2 coding sequence.

27-29. (canceled)

30. The method of claim 1 wherein the glucose is present in the inducing feed composition in an amount comprising about 60% wt/wt, the sophorose is present in the inducing feed composition in an amount comprising about 12 g/L, the nucleic acid molecule encoding the hydrophobin is a hfb2 coding sequence, the hfb2 coding sequence is operably linked to the cbh1 promoter whereby hydrophobin expression is under the control of the cbh1 promoter, and the Trichoderma comprises a Trichoderma reesei having one or more of Trichoderma cbh1, cbh2, egl1, egl5 and egl2 coding sequences deleted or disrupted.

31. A method for producing a hydrophobin encoded by a gene under the control of an inducible promoter comprising the steps of:
(a) incubating a mixture comprising between about 5% to about 75% glucose and a cellulase preparation at a temperature and for a sufficient time to produce an inducing feed composition comprising sophorose in a concentration ranging from 2 g/L to 25 g/L, gentiobiose in a concentration ranging from 35 g/L to 60 g/L, and glucose; and
(b) culturing a host cell comprising a nucleotide sequence encoding a hydrophobin under the control of a sophorose-inducible promoter or a gentiobiose-inducible promoter with said inducing feed composition, in an amount effective to induce the production of hydrophobin.


SEQUENCE LISTING (2 sequences)

SEQ ID NO: 1 - DNA, 667 bp, Trichoderma reesei
  misc_feature: hydrophobin II gene (hfb2)
cacattcactcaactcctctttctcaactctccaaacacaaacattctttgttgaataccaaccatcaccacctttcaagatgcagttcttcgccgtcgccctcttcgccaccagcgccctggctgctgtctgccctaccggcctcttctccaaccctctgtgctgtgccaccaacgtcctcgacctcattggcgttgactgcaagacccgtatgttgaattccaatctctgggcatcctgacattggacgatacagttgacttacacgatgctttacagctaccatcgccgtcgacactggcgccatcttccaggctcactgtgccagcaagggctccaagcctctttgctgcgttgctcccgtggtaagtagtgctcgcaatggcaaagaagtaaaaagacatttgggcctgggatcgctaactcttgatatcaaggccgaccaggctctcctgtgccagaaggccatcggcaccttctaaagcaatggcttgctttactgccggcagtctttgagaactctgggctcacaaaagacgacttgcatgtatcatgggggctcgcaaatgggaggatttggaggggattgaggctgggtttggcctattagaggattgcataatggaagatttgcgagcaggacatagacgtatctagagttctagt

SEQ ID NO: 2 - protein, 86 aa, Trichoderma reesei
  misc_feature: translated hydrophobin II gene (hfb2)
MQFFAVALFATSALAAVCPTGLFSNPLCCATNVLDLIGVDCKTPTIAVDTGAIFQAHCASKGSKPLCCVAPVADQALLCQKAIGTF